(19) United States
(12) Patent Application Publication
(10) Pub. No.: US 2013/0253399 A1
(43) Pub. Date: Sep. 26, 2013

(54) Harness

(71) Applicant: Barbara Thompson, Linconshire (GB)

(72) Inventor: Barbara Thompson, Linconshire (GB)

(21) Appl. No.: 13/892,654

(22) Filed: May 13, 2013

Related U.S. Application Data
(63) Continuation of application No. 13/059,800, filed on Aug. 9, 2011, now abandoned.

(30) Foreign Application Priority Data
Aug. 19, 2008 (GB) 0815045.0

(51) Int. Cl. A61F 5/058

(52) U.S. Cl. CPC A61F 5/05808
USPC 602/19

(57) ABSTRACT

A harness (1; 33) for supporting a fractured clavicle, the harness comprising a first part (3; 35) that is securable about the waist of a user and extends in use to cover at least a portion of the user's back, and first and second straps (13, 15; 39, 41) secured to that portion of the first part (3, 35) that covers at least a portion of the user's back in use, wherein the first and second straps (13, 15; 39, 41) are capable of begin looped over the user's shoulders, under the user's arms and can be secured in position, and at least one of said first and second straps includes a support member (19; 45) that is configured to locate beneath at least part of the fractured clavicle to support and align the component parts of the fractured clavicle whilst the clavicle heals.

### CROSS-REFERENCE TO RELATED APPLICATIONS

This present application is a continuation of U.S. Patent application Ser. No. 13/059,800, filed on Aug. 9, 2011, which claims the benefit of International Application Serial No. PCT/EP2009/060752, filed on Aug. 19, 2009, which claims priority to United Kingdom patent application No. GB 0815045.0, filed on Aug. 19, 2008, the contents of which are hereby incorporated by reference in their entireties.

### FIELD OF THE INVENTION

This invention relates to a harness, for example to a harness for stabilizing fractured clavicles to enable the fracture to heal. In a particularly preferred embodiment the harness further comprises a sling for supporting the arm.

### BACKGROUND OF THE INVENTION

A number of harnesses for fractured clavicles (otherwise known as collarbones) are currently offered for sale. These devices are generally designed to stabilise a fractured clavicle whilst the fracture heals.

One such device is the FIG. 8 Clavicle Splint offered for sale by Ossur. This device comprises a buckle that lies between the shoulder blades of a user; a first strap that extends from the buckle over one shoulder of the patient, under the patient's arm and couples to the buckle, and a second strap that extends from the buckle over the other shoulder of the patient, under the patient's other arm and couples to the buckle.

One problem with this device is that it is relatively uncomfortable to wear because the buckle tends to ride up and bear against the back of the wearer's neck. Once this occurs it is very difficult and typically quite painful for the user to pull the buckle back down into the correct position, Another problem is that it is very difficult for a user to put on the device without assistance.

Another device that can be used as a clavicle harness is the Premium Posture Correction Support Brace offered for sale by, inter alia, The Back Care Warehouse (see: http:www.thebackcamwarehouse.co.uk/acatalog/PostureCorrectionShoulderBraces.html). This device is similar in function to the Ossur device, but instead of a buckle worn between the shoulder blades this device includes an elasticated strap that is worn about the waist and to which the straps that extend over the shoulders of the user are coupled. By avoiding the provision of a buckle this device does tend to dig in to a lesser extent to the back of the user's neck, but a problem with this device is that the waist strap tends to rise up during use, whereupon tension in the over-shoulder straps is lost and the device ceases to function effectively as a means to stabilise the fractured clavicle.

Another problem associated with such devices is that in some instances they do not adequately support the fractured clavicle so that the component parts are correctly aligned when healing occurs. If this should happen it is often the case that the only course of corrective treatment available is to surgically intervene by re-breaking the clavicle, correctly aligning the component parts and then rejoining them by means of one or more metal plates. As all surgical interventions carry some risk and are generally quite expensive, it would advantageous if the need for surgical intervention could be reduced.

Another problem that affects both the Ossur device and, the device offered for sale by The Back Care Warehouse is that the over-shoulder straps of each device must be tensioned to such an extent that they tend to adversely affect neurovascular structures in the underarm area of the body of the wearer. In particular, such devices can cause a circulatory restriction in this area of the body, leaving the wearer with a loss of sensation or tingling in the arm. This problem is not limited to braces that are particularly designed for supporting fractured clavicles, but instead afflicts all manner of upper body braces.

The present invention has been devised with the above problems in mind.

### SUMMARY OF INVENTION

To this end, an embodiment of the present invention provides a harness for supporting a fractured clavicle, the harness comprising a first part that is securable about the waist of a user and extends in use to cover at least a portion of the user's back, and first and second straps secured to that portion of the first part that covers at least a portion of the user's back in use, Wherein the first and second straps are capable of being looped over the user's shoulders, and under the user's arms before being secured in position, and at least one of said first and second straps includes a support member that is capable of locating beneath at least part of the fractured clavicle to support and align the component parts of the fractured clavicle whilst the clavicle heals.

In one envisaged implementation, each of said straps may include a said support member. In a preferred arrangement, said support member is configured to lessen the effect of the strap on neurovascular structures under the user's arm. The support member may be configured to reduce the extent to which the strap bears on neurovascular structures under the user's arm, For example, the support member may be configured to space the strap from the neurovascular structures under the user's arm.

The support member may comprise a resiliently compressible body.

The first part may comprise a band that is securable about a portion of the patient's torso. In one arrangement, the band is configured, when secured about the user, to cover substantially all of the patient's abdomen.

In one embodiment, the first and second straps are configured to be secured behind the user. For example said first and second straps may be configured to be secured to said first part behind the user to secure the straps in position.

In another embodiment, said first and second straps are configured to be secured in front of the user. For example, said first and second straps may be configured to be secured to one another in front of the user. In an envisaged implementation, each strap has a free end, one free end including a first part of a two-part closure mechanism and the other free end including a second part of said mechanism, said first and second parts being capable of being coupled one to the other to join said one free end to said other free end.

The harness may comprise a pair of slotted brackets located behind the user in use and through which said straps extend before being secured in position in front of the user.

The harness may further comprise a chest strap for resisting relative movement between the straps in use. For example, one end of said chest strap may be secured to one strap and another end of said chest strap may be secured to the other strap.

Another embodiment of the present invention relates to a harness for supporting a fractured clavicle, the harness comprising as first part that is securable about the waist of a user and extends in use to cover at least a portion of the user's back, and a strap secured to that portion of the first part that covers at least a portion of the user's back in use, wherein the strap is capable of being looped over the shoulder of the user that includes the fractured clavicle, under the user's arm and can be secured in position, said strap further comprising a support member that is capable of locating beneath at least part of the fractured clavicle to support and align the component parts of the fractured clavicle whilst the clavicle heals.

The harness may comprise a sling securable about at least the lower arm of the user, the sling being attachable to said harness. The sling may include a strap that is capable of being passed over the shoulder of the user and secured to said first part to support the arm of the user in front of their torso. The sling may include a strap that is attachable to a strap of the harness and an attachment mechanism for coupling a part of the sling into which the user inserts their arm to the harness.

In accordance with another embodiment of the present invention there is provided an upper body brace comprising a strap that extends over the shoulder and under the arm of a subject, the strap including a support member that spaces the strap from the body of the subject to reduce the extent to which the strap bears on neurovascular structures under the user's arm. An advantage of this arrangement is that by reducing the extent to which the strap bears on neurovascular structures under the user's arm, so the likelihood of the brace causing tingling or loss of sensation can be reduced.

Other features, embodiments and advantages of the present invention will be apparent from the following detailed description.

### BRIEF DESCRIPTION OF THE DRAWINGS

Various aspects of the teachings of the present invention, and arrangements embodying those teachings, will hereafter be described by way of illustrative example with reference to the accompanying drawings, in which:

FIG. 1 is a plan view of the harness according to an embodiment of the present invention with the various components shown disconnected;

FIG. 2 is a front view of the harness with the various components connected together;

FIG. 3 is a rear view of the harness mounted on a cardboard support;

FIG. 4 is a front view of the harness being worn by a user;

FIG. 5 is an enlarged view of a support member;

FIG. 6 is a rear view of the harness being worn by a user;

Fig, 7 is a front view of the harness and sling;

FIG. 8 is a side view of the harness and sling;

FIG. 9 is a front view of a harness according to another embodiment of the present invention;

FIG. 10 is a rear view of the harness shown in FIG. 9;

FIG. 11 is an enlarged view of the rear of the harness; and

FIG. 12 is a front view of the harness shown in FIG. 9 in use with a sling.

### DETAILED DESCRIPTION OF PREFERRED EMBODIMENTS

Preferred embodiments of the present invention will now be described with particular reference to a harness for the support of a fractured clavicle, It should be noted, however, that this is merely one of many different applications for the teachings of the present invention, and that the devices described herein may be used for applications other than the support of a fractured clavicle. As such the following description should be read as being merely illustrative of the teachings of the present invention, and not as a limitation of the scope thereof.

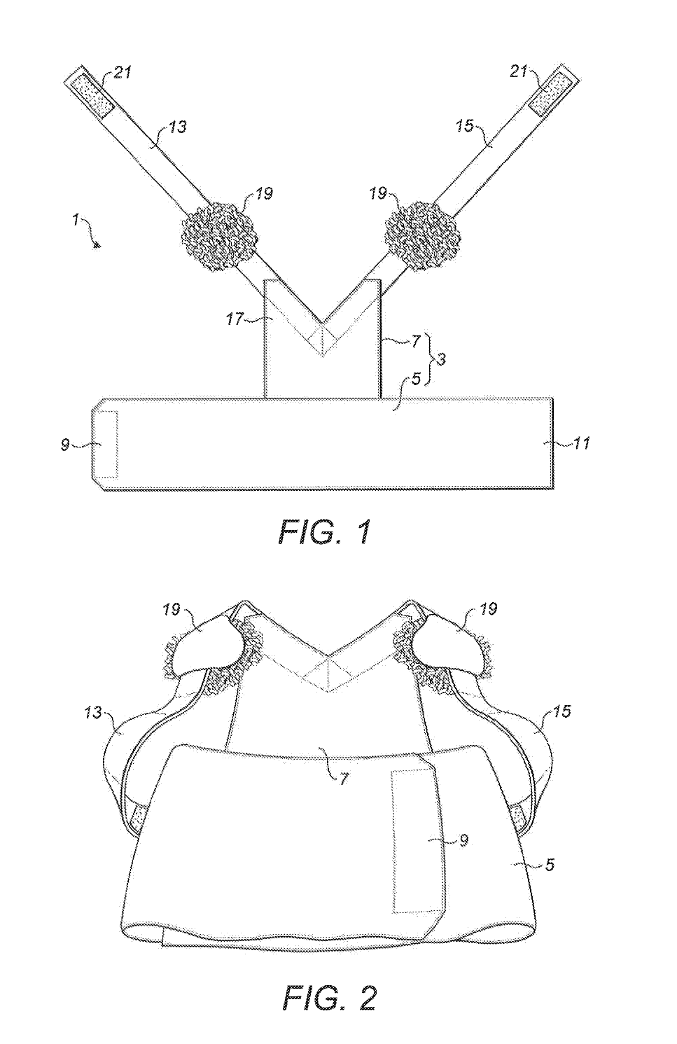

Referring now to FIG. 1, the harness 1 of this embodiment comprises a first part 3 that comprises a band 5 that is adapted to be secured about the body of a user and a web 7 that is fixed at one end to the band 5. in the preferred arrangement the band is securable about a portion of the user's torso. In a particularly preferred arrangement the band is configured to cover substantially all of the user's abdomen (i.e. generally from the user's hips to their ribs) when it is secured about the user. This latter arrangement is particularly advantageous as it makes it quite difficult for the band to rise up the body in use.

One free end 9 of the band 5 includes a strip of mechanical fasteners (such as Velcro™, for example) so that the free end 9 can be wound round the user's body to overlap the other free end 11 of the band before being secured to the band by means of the mechanical fastener.

In this embodiment first and second straps 13, 15 are coupled to the web in the vicinity of an end 17 of the web 7 remote from the end that is coupled to the band 5. In another embodiment, only a single strap is provided. The web covers at least a portion of the user's back when the harness 1 is worn, and web end 17 may, as shown, be cut-away to form a V so that the harness does not impinge on the back of the user's neck in use.

In this embodiment each of the straps 13, 15 carry a support member 19 (the function of which will later be described) and the free ends of the straps each include a strip of mechanical fastener 21 (for example Velcro) so that they can readily be secured in place when the harness is in use. In another embodiment only one of the straps (in particular the strap that loops over the shoulder with the fractured clavicle) may carry a support member.

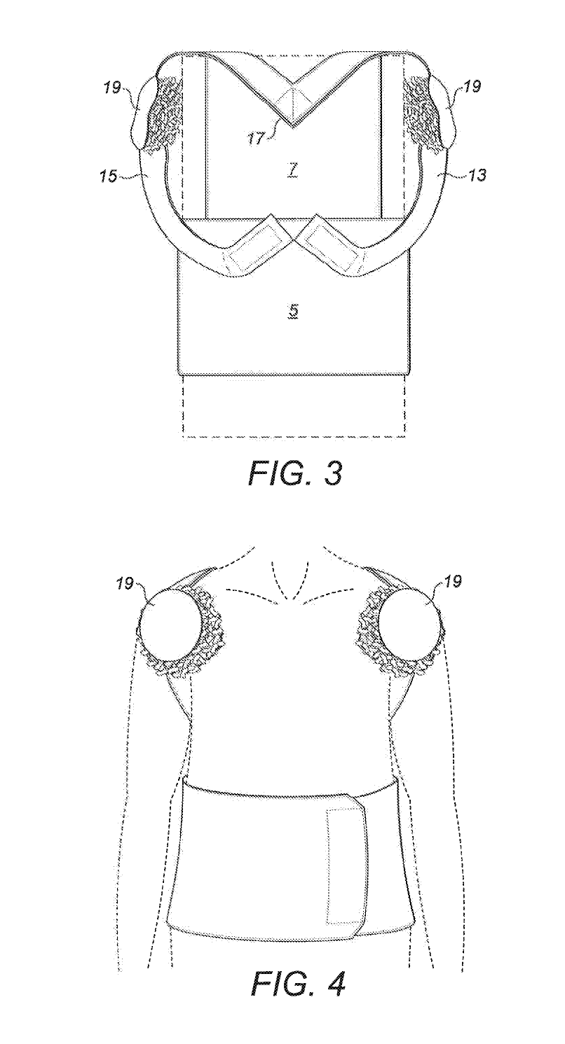

Referring now to FIG. 2, in use the band 5 is secured about a portion of the torso of a user so that the web 7 lies behind the user. The straps 13, 15 are then looped over respective arms of the user, passed under the arms and—in this embodiment then secured to the rear of the first part 3, for example to the rear of the band and/or the web 7. This arrangement is shown more clearly in FIG. 3.

As shown in FIG. 4, when the harness is worn one strap 13, 15 loops over each of the user's shoulders and under the arm before being secured behind the user to the first part 3. When the straps are secured in place the support members 19 bear on the chest of the user at a point just below the user's clavicles and help to support the fractured clavicle in correct alignment whilst it is healing. In a particularly preferred arrangement the support members are moveable on each of the straps so that they can be best positioned to provide support to the user's clavicles.

Each support members 19 also functions to reduce the extent to which the associated strap bears on the user's body, in particular on the underarm region of the user's body. In one configuration the support members function to space the associated straps from the user's body to reduce the extent to which the straps bear on the user's body.

The advantage of this arrangement is that the strap tends to bear—to a lesser degree—on neurovascular structures under the user's arm and as a consequence the harness according to the teachings of this invention is less likely to cause the numbness and tingling that can be characteristic of previously proposed devices,

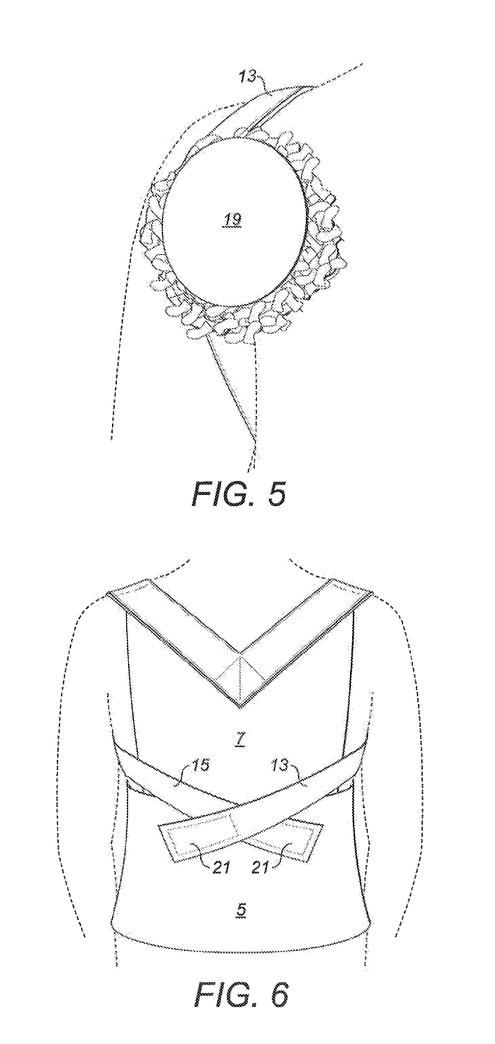

FIG. 5 is an enlarged view of a support member 19. As shown the support member comprises a pad of compressible material. In the preferred embodiment the support member is generally hemi-spherical with a generally planar surface that is worn facing away from the body and a curved surface that is worn facing the body. The planar and curved surfaces define an internal void that contains, and is preferably filled with a resilient material to enable the pad to compress. In the preferred arrangement the strap passes through a channel in the support member so that the resilient material lies between the strap and the user, and in this arrangement the strap presses the material against, the user's body to support and align the clavicle whilst it heals.

FIG. 6 is a rear view of the harness being worn by a user. As shown the first and second straps 13, 15 have been looped over the shoulders of the user, passed under each arm and have been secured to the band 5 and/or web 7. The straps are tensioned by the user as the user puts on the harness and function to urge the shoulder blades of the user backwards towards the user's back. This arrangement stabilises the user's shoulders, in particular the fractured clavicle, so that the clavicle can heal and knit back together.

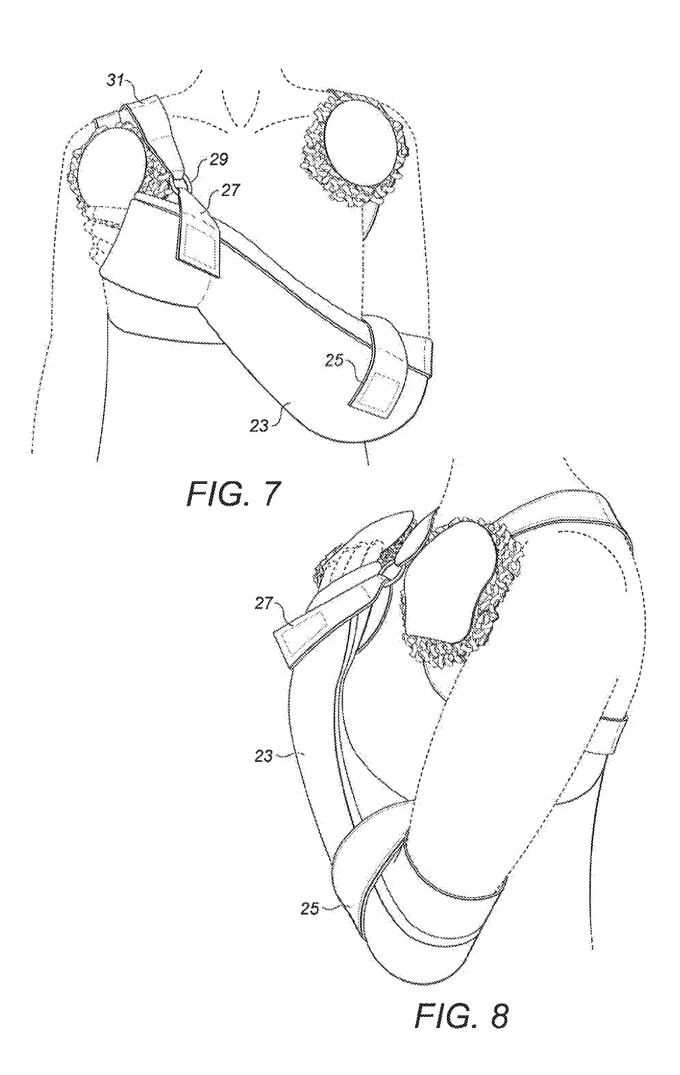

FIGS. 7 and 8 are front and side views, respectively, of the harness together with an optional sling 23 that is useful, particularly shortly after a fracture has occurred, as a support for the arm of the user that is adjacent the fractured clavicle. The sling comprises a length of material (preferably a relatively soft material) that can be fashioned into a tube about the arm of the user and secured in place by first and second straps 25, 27 that bear a mechanical fastener, such as Velero™. The second strap, proximate the user's hand in use, is coupled by way of a. coupling 29 to a third strap 31 that extends over the user's good shoulder and is provided with a mechanical fastener on a free end thereof (not shown) so that it can be secured to the first part 3, for example to the web 7.

As will be appreciated by persons skilled in the art, the sling functions to support the arm adjacent the fractured clavicle and thereby helps to immobilise the arm and clavicle as well as to reduce the strain on the fractured clavicle whilst it heals.

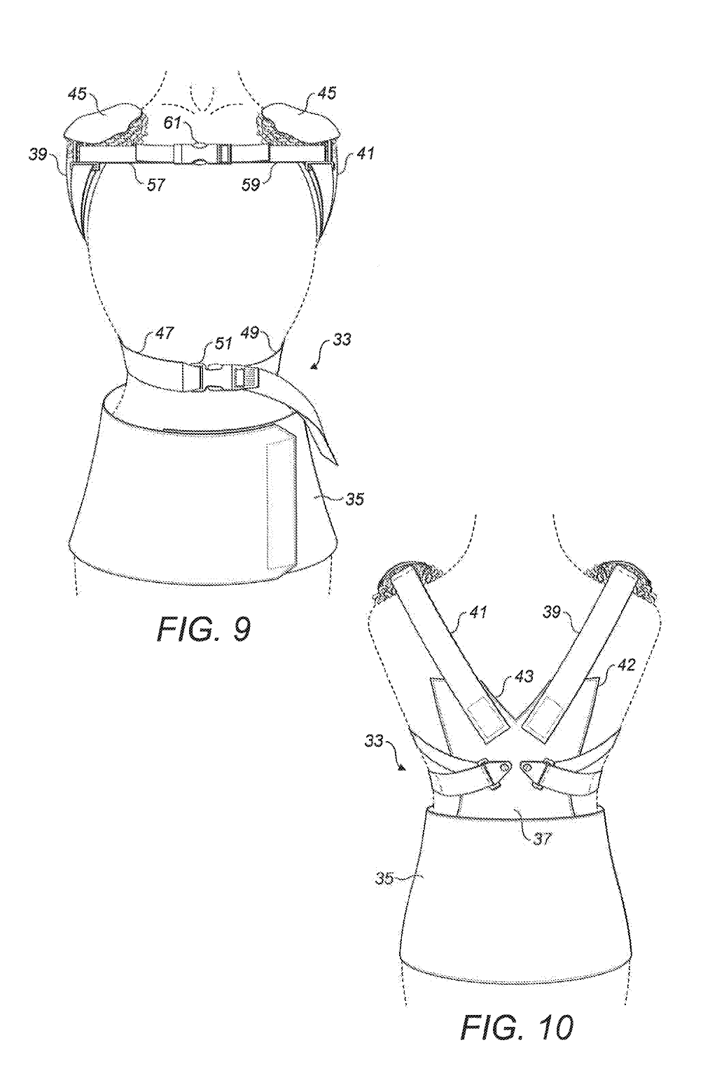

FIGS. 9 and 10 show front and rear views, respectively, of a harness 33 according to a second embodiment of the present invention. The harness of this embodiment is similar to that of the first embodiment in that it comprises a first part which comprises a band 35 that is configured to be secured around the body/abdomen of a user and a web 37 that is fixed at one end to the band 35. As before, the web and band may be integrally formed, or separately formed and joined one to the other.

In this embodiment a pair 39, 41 of straps are joined to an end 42 of the web 37 remote from the band (which end may include a cut-out portion 43 as previously described). The straps 39, 41 each carry a support member 45 that is identical in form and function to the support member 19 of the first embodiment, and each have a free end 47, 49 (shown secured one to the other in FIG. 9).

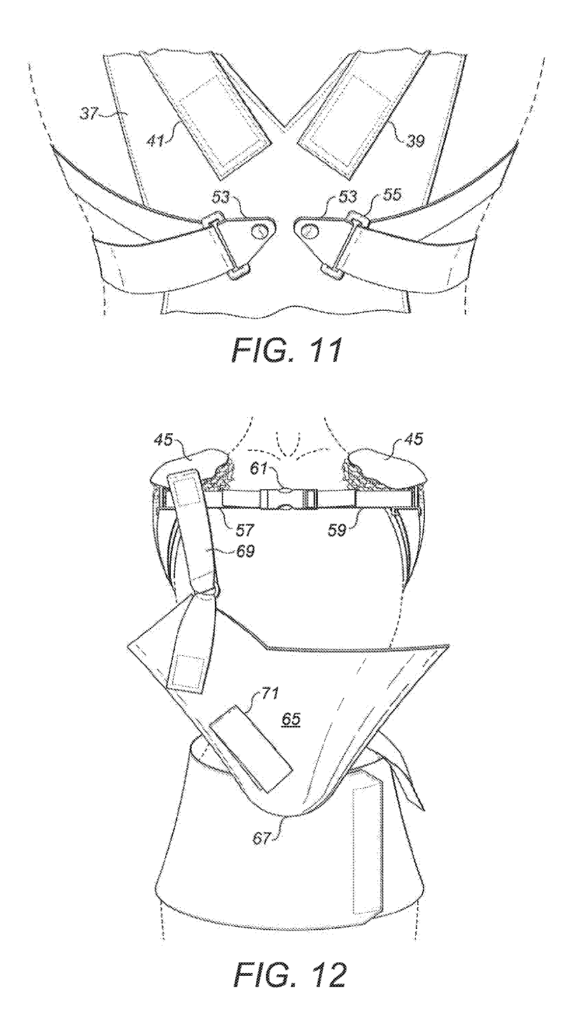

Referring now to FIGS. 9 and 11, the harness 33 of this embodiment differs from that of the first embodiment in that the free ends of the straps 13, 15 of the first embodiment secure to the web 7 behind the user, whereas the strap free ends 47, 49 of this embodiment are secured (preferably one to the other) in front of the user (as shown in FIG. 9).

In the preferred arrangement the free ends of the straps carry respective parts of a two part closure mechanism 51, thereby enabling one free end to be secured to the other. A variety of such closure mechanisms are known in the art, and any such suitable mechanism may be employed. In another arrangement, the free ends of the straps may include a mechanical fastener (such as Velcro) and be securable to the band 35 in front of the user, or indeed or the user's clothing. One advantage of securing the straps in front of the user is that the harness can more easily be put on by a user without the assistance of another.

To enable the straps to be secured in front of the user, the harness includes (as shown in FIG. 11) first and second brackets 53 that are secured to the web 37 below the end to which the straps 39, 41 are secured. The brackets each include a slot 55 through which respective free ends of the straps are fed before being passed round in front of the user for securing. When the harness 33 is being worn, the strap or straps each extend from the end 42 of the web 37, over a shoulder of the user, under the user's arm, through the slot 55 in the bracket 53 and back under the user's arm for securing in front of the user.

As shown in FIGS. 9 and 12, the harness may optionally comprise a chest strap that consists of a first part 57 secured to one strap 39 and a second part 59 secured to the other strap 41, the first and second parts each having a free end that carries a respective complementary part of a two-part closure mechanism 61. The free ends of the first chest strap part and the second chest strap part can be secured together (as shown in FIGS. 9 and 12) to further resist relative movement between the straps 39, 41 and thereby further stabilise the clavicle of the user.

Referring again to FIG. 12 of the drawings, there is shown a sling 63 that may be used with the harness of either embodiment.

The sling 63 comprises first and second similarly shaped panels (only the outer one 65 of which is visible) that have been joined together to form a generally triangular portion 67 that opens towards the user (along the upper edge as shown in the drawings) so that a user can insert their arm into the sling. The sling has a curved corner 65 that supports the user's elbow, and is coupled to the harness by means of a mechanical fastener (not visible) that is attached to the inner panel (not visible) and arranged to attach to the harness, and by means of a strap 69 that couples one end of the sling to one of the harness straps (for example by means of a mechanical fastener).

Note that whilst the mechanical fastener on the inner panel is not visible, an identical fastener 71 on the outer panel is shown in FIG. 12, the outer panel fastener being used to couple the sling to the harness when the user's other arm is supported by the sling.

As with the other sling disclosed herein, the sling shown in FIG. 12 functions to support the arm adjacent the fractured clavicle and thereby helps to immobilise the arm and clavicle as well as to reduce the strain on the fractured clavicle whilst it heals. Importantly, the arm is supported from below (by means of the mechanical fastener on the inner panel) and as such the arm does not bear on the neck of the user as is the case with existing slings.

It will be appreciated that whilst various aspects and embodiments of the present invention have heretofore been described, the scope of the present invention is not limited to the particular arrangements set out herein and instead extends to encompass all arrangements, combinations of features and/or embodiments, and modifications and alterations thereto, which fall within the scope of the appended claims.

For example, whilst the slings are described herein as being for use with a harness of the type described, it will be appreciated that the slings need not necessarily be limited to being used with the harnesses described herein.

It is well known in the art that a typical sling includes a strap that passes round the neck of the wearer, and such slings may not be suitable for patients that are unable to tolerate the added weight of an arm supported in a sling passing round their neck. For example, if a patient has damaged their back and broken their arm, then a sling which suspended the patient's arm from the patient's neck would bring additional pressure to bear on the neck, to the detriment of the back.

In such circumstances, slings of the type described herein where the arm is supported from underneath (rather than being suspended from the neck) would be beneficial. To that end, another embodiment of the present invention relates to a sling system for supporting a user's arm, the system comprising a web that defines a pocket into which at least part of a user's arm may be inserted, a first fastener carried by the subject, and a second fastener part coupled to said web, wherein said second fastener part can be attached to said first fastener part to support said web on the subject.

In one illustrative arrangement the first fastener part may comprise part of an upper body brace. The second fastener part may comprise hooks of a mechanical hook-and-loop fastener system. The first fastener part may comprise loops of a mechanical hook-and-loop fastener system, or in the alternative a material to which hooks of a mechanical hook-and-loop fastener system can be attached. The first fastener part could comprise, for example, an item of clothing that can be worn by a subject.

It will also be apparent, and indeed has already been mentioned. above that the teachings of the present invention are not limited to harnesses for the treatment of fractured clavicles. Rather, the teachings of the present invention may be applied to any upper body brace where a part of the brace exerts a force on the body of the wearer that restricts circulation, and to that end a preferred embodiment of the present invention relates to an upper body brace that includes a support member which is configured to space a part of said brace from the body of the subject to reduce the extent to which that part bears on the user's body. In one envisaged arrangement the support member is attached to a strap that passes over the shoulder and under the arm of a subject, and the support member functions to space the strap from the body of the patient to reduce the extent to which the strap bears upon the underarm region of the patient's body.

In another arrangement, whilst in one embodiment the band and web are fixedly coupled together (for example, sewed together) and the first and second straps are fixedly coupled to the web 7 (for example, sewed to the web 7), in an alternative arrangement the web and/or straps could be provided with a mechanical connector (such as Velcro) that the harness can be completely disassembled for easy storage.

It is also the case that whilst in one embodiment both first and second straps are provided, in an alternative arrangement only a first or second strap may be provided. In yet another arrangement, both the first and second straps may be provided, but only one of the straps may include a support member.

Lastly, it should also be noted that whilst the accompanying claims set out particular combinations of features described herein, the scope of the present invention is not limited to the particular combinations hereafter claimed, but instead extends to encompass any combination of features herein disclosed.

What is claimed is:

1. A harness for supporting a fractured clavicle, the harness comprising a first part that is securable about the waist of a user and extends in use to cover at least a portion of the user's back, and first and second straps secured to that portion of the first part that covers at least a portion of the user's back in use, wherein the first and second straps are capable of being looped over the user's shoulders, and under the user's arms before being secured in position, and at least one of said first and second straps includes a support member that is capable of locating beneath at least part of a fractured clavicle to support and align the component parts of the fractured clavicle whilst the clavicle heals.

2. A harness according to claim 1, wherein each of said straps includes a said support member.

3. A harness according to claim 1, wherein the support member is configured to lessen the effect of the strap on neurovascular structures under the user's arm.

4. A harness according to claim 3, wherein the support member is configured to reduce the extent to which the strap bears on neurovascular structures under the user's arm.

5. A harness according to claim 4, wherein the support member is configured to space the strap from the neurovascular structures under the user's arm.

6. A harness according to claim 1, wherein the support member comprises a resiliently compressible body.

7. A harness according to claim 1, wherein the first part comprises a band that is securable about a portion of the patient's torso.

8. A harness according to claim 7, wherein the band is configured, when secured about the user, to cover substantially all of the patient's abdomen.

9. A harness according to claim 1, wherein said first and second straps are configured to be secured behind the user.

10. A harness according to claim 9, wherein said first and second straps are configured to be secured to said first part behind the user to secure the straps in position.

11. A harness according to claim 1, wherein said first and second straps are configured to be secured in front of the user.

12. A harness according to claim 11, wherein said first and second straps are configured to be secured to one another in front of the user.

13. A harness according to claim 12, wherein each strap has a free end, one free end including a first part of a two-part closure mechanism and the other free end including a second part of said mechanism, said first and second parts being capable of being coupled one to the other to join said one free end to said other free end.

14. A harness according to claim 11, wherein said harness comprises a pair of slotted brackets located behind the user in use and through which said straps extend before being secured in position in front of the user.

15. A harness according to claim 1, further comprising a chest strap for resisting relative movement between the straps in use.

16. A harness according to claim 15, wherein one end of said chest strap is secured to one strap and another end of said chest strap is secured to the other strap.

17. A harness for supporting a fractured clavicle, the harness comprising a first part that is securable about the waist of a user and extends in use to cover at least a portion of the user's back, and a strap secured to that portion of the first part that covers at least a portion of the user's back in use, wherein the strap is capable of being looped over the shoulder of the user that includes the fractured clavicle, under the user's arm and can be secured in position, said strap further comprising a support member that is capable of locating beneath at least part of the fractured clavicle to support and align the component parts of the fractured clavicle whilst the clavicle heals.

18. A harness according to claim 17, further comprising a sling securable about at least the lower arm of the user, the sling being attachable to said harness.

19. A harness according to claim 18, wherein the sling includes a strap that is capable of being passed over the shoulder of the user and secured to said first part to support the arm of the user in front of their torso.

20. A harness according to claim 18, wherein the sling includes a strap that is attachable to a strap of the harness and an attachment mechanism for coupling a part of the sling into which the user inserts their arm to the harness.

21. An upper body brace that includes a support member which is configured to space a part of said brace from the body of the subject to reduce the extent to which that part bears on the user's body.

22. A brace according to claim 21, wherein the support member is attached to a strap that passes over the shoulder and under the arm of a subject, and the support member functions to space the strap from the body of the patient to reduce the extent to which the strap bears upon the underarm region of the patient's body

23. A sling system for supporting a user's arm, the system comprising a web that defines a pocket into which at least part of a user's arm may be inserted, a first fastener carried by the subject, and a second fastener part coupled to said web, wherein said second fastener part can be attached to said first fastener part to support said web on the subject.
